(19) United States
(12) Patent Application Publication
(10) Pub. No.: US 2022/0226114 A1
(43) Pub. Date: Jul. 21, 2022

(54) BALLOON VALVULOPLASTY CATHETER WITH IVUS

(71) Applicant: BOSTON SCIENTIFIC SCIMED, INC., Maple Grove, MN (US)

(72) Inventors: Dongming Hou, Plymouth, MN (US); Tim O'Connor, Galway (IE); Wenguang Li, Los Gatos, CA (US); Anming He Cai, San Jose, CA (US)

(73) Assignee: BOSTON SCIENTIFIC SCIMED, INC., Maple Grove, MN (US)

(21) Appl. No.: 17/568,217

(22) Filed: Jan. 4, 2022

Related U.S. Application Data
(60) Provisional application No. 63/138,899, filed on Jan. 19, 2021.

(51) Int. Cl. A61F 2/24; A61B 8/12; A61B 8/08; A61M 29/02

(52) U.S. Cl. CPC A61F 2/2433; A61B 8/12; A61M 2029/025; A61M 29/02; A61M 2205/32; A61B 8/0883

(57) ABSTRACT

A balloon valvuloplasty catheter may include an elongate shaft having a guidewire lumen and a device lumen extending longitudinally therein, an expandable balloon secured to a distal portion of the elongate shaft, and an intravascular ultrasound catheter slidably disposed within the device lumen. The device lumen is in fluid communication with an interior of the expandable balloon. A method of preparing a native aortic heart valve of a patient's heart for transcatheter aortic valve replacement may include using the balloon valvuloplasty to observe via intravascular ultrasound and evaluate a position of the native leaflets relative to the left and right coronary artery ostia to determine if the native leaflets block the left coronary artery ostium and/or the right coronary artery ostium when the expandable balloon is inflated.

### CROSS-REFERENCE TO RELATED APPLICATIONS

This application claims the benefit of priority of U.S. Provisional Application No. 63/138,899 filed Jan. 19, 2021, the entire disclosure of which is hereby incorporated by reference.

### TECHNICAL FIELD

The present disclosure pertains to medical devices, and methods for manufacturing and/or using medical devices. More particularly, the present disclosure pertains to a balloon valvuloplasty catheter for use in heart valves.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In a first example, a balloon valvuloplasty catheter may comprise an elongate shaft having a guidewire lumen and a device lumen extending longitudinally therein; an expandable balloon secured to a distal portion of the elongate shaft; and an intravascular ultrasound catheter slidably disposed within the device lumen. The device lumen may be in fluid communication with an interior of the expandable balloon.

In addition or alternatively to any example disclosed herein, the intravascular ultrasound catheter is configured to image tissue surrounding the expandable balloon when the expandable balloon is in an expanded configuration.

In addition or alternatively to any example disclosed herein, the elongate shaft includes an inflation lumen in fluid communication with the interior of the expandable balloon.

In addition or alternatively to any example disclosed herein, the device lumen defines at least a portion of the inflation lumen.

In addition or alternatively to any example disclosed herein, the device lumen includes a proximal seal configured to engage an outer surface of the intravascular ultrasound catheter.

In addition or alternatively to any example disclosed herein, the intravascular ultrasound catheter includes an ultrasound transducer disposed proximate a distal end of the intravascular ultrasound catheter.

In addition or alternatively to any example disclosed herein, the ultrasound transducer is configured to translate longitudinally within the expandable balloon.

In addition or alternatively to any example disclosed herein, the device lumen terminates within the interior of the expandable balloon.

In addition or alternatively to any example disclosed herein, a first radiopaque marker is disposed adjacent a proximal end of the expandable balloon and a second radiopaque marker is disposed adjacent a distal end of the expandable balloon.

In addition or alternatively to any example disclosed herein, a method of preparing a native aortic heart valve of a patient's heart for transcatheter aortic valve replacement may comprise:

advancing a guidewire percutaneously through the native aortic heart valve and into a left ventricle of the patient's heart;

advancing a balloon valvuloplasty catheter over the guidewire to a position adjacent the native aortic heart valve;

wherein the balloon valvuloplasty catheter comprises:
an elongate shaft having a guidewire lumen and a device lumen extending longitudinally therein;
an expandable balloon secured to a distal portion of the elongate shaft; and
an intravascular ultrasound catheter slidably disposed within the device lumen, wherein the device lumen is in fluid communication with an interior of the expandable balloon;

positioning the expandable balloon within the native aortic heart valve;

imaging a left coronary artery ostium, a right coronary artery ostium, and native leaflets of the native aortic heart valve using the intravascular ultrasound catheter;

inflating the expandable balloon within the native aortic heart valve; and

observing via intravascular ultrasound a position of the native leaflets relative to the left coronary artery ostium and the right coronary artery ostium.

In addition or alternatively to any example disclosed herein, the method may comprise adjusting a position of the intravascular ultrasound catheter within the expandable balloon by sliding the intravascular ultrasound catheter axially relative to the elongate shaft.

In addition or alternatively to any example disclosed herein, the method may comprise evaluating the position of the native leaflets relative to the left coronary artery ostium and the right coronary artery ostium to determine if the native leaflets block the left coronary artery ostium and/or the right coronary artery ostium when the expandable balloon is inflated.

In addition or alternatively to any example disclosed herein, the method may comprise imaging the native aortic heart valve using the intravascular ultrasound catheter to determine a size of the native aortic heart valve.

In addition or alternatively to any example disclosed herein, imaging the native aortic heart valve using the intravascular ultrasound catheter occurs while inflating the expandable balloon within the native aortic heart valve.

In addition or alternatively to any example disclosed herein, imaging the native aortic heart valve using the intravascular ultrasound catheter occurs while the expandable balloon is fully inflated within the native aortic heart valve.

In addition or alternatively to any example disclosed herein, a method of repairing a native aortic heart valve of a patient's heart may comprise:

advancing a guidewire percutaneously through the native aortic heart valve and into a left ventricle of the patient's heart;

advancing a balloon valvuloplasty catheter over the guidewire to a position adjacent the native aortic heart valve;

wherein the balloon valvuloplasty catheter comprises:
an elongate shaft having a guidewire lumen and a device lumen extending longitudinally therein;
an expandable balloon secured to a distal portion of the elongate shaft; and
an intravascular ultrasound catheter slidably disposed within the device lumen, wherein the device lumen is in fluid communication with an interior of the expandable balloon;

positioning the expandable balloon within the native aortic heart valve;

imaging a left coronary artery ostium, a right coronary artery ostium, and native leaflets of the native aortic heart valve using the intravascular ultrasound catheter;

inflating the expandable balloon within the native aortic heart valve and observing via intravascular ultrasound a position of the native leaflets relative to the left coronary artery ostium and the right coronary artery ostium;

evaluating the position of the native leaflets relative to the left coronary artery ostium and the right coronary artery ostium to determine if the native leaflets block the left coronary artery ostium and/or the right coronary artery ostium when the expandable balloon is inflated;

removing the balloon valvuloplasty catheter while maintaining the guidewire in position within the left ventricle of the patient's heart;

advancing a delivery device over the guidewire to the native aortic heart valve; and

thereafter, deploying a replacement aortic heart valve implant within the native aortic heart valve using the delivery device such that neither the left coronary artery ostium nor the right coronary artery ostium is blocked by the native leaflets when the replacement aortic heart valve implant is deployed.

In addition or alternatively to any example disclosed herein, if one or both of the left coronary artery ostium and the right coronary artery ostium is blocked by the native leaflets when the expandable balloon is inflated, deployment of the replacement aortic heart valve implant is abandoned.

In addition or alternatively to any example disclosed herein, the method may comprise imaging the native aortic heart valve using the intravascular ultrasound catheter while the expandable balloon is fully inflated to determine a size of the native aortic heart valve.

In addition or alternatively to any example disclosed herein, the method may comprise selecting the replacement aortic heart valve implant based on the size of the native aortic heart valve as determined by imaging the native aortic heart valve using the intravascular ultrasound catheter.

In addition or alternatively to any example disclosed herein, the method may comprise loading the replacement aortic heart valve implant selected into the delivery device.

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically illustrates an example configuration of a heart;

FIG. 2 illustrates an example replacement aortic valve implant disposed in the heart of FIG. 1;

FIG. 3 schematically illustrates another example configuration of a heart;

FIG. 4 illustrates the example replacement aortic valve implant disposed in the heart of FIG. 3;

FIG. 5 illustrates aspects of a balloon valvuloplasty catheter;

FIGS. 6-7 illustrate aspects of using the balloon valvuloplasty catheter within a heart;

FIG. 8 is a block diagram showing a portion of a method of preparing a native aortic heart valve of the heart for transcatheter aortic valve replacement and/or the method of repairing the native aortic heart valve; and

FIGS. 9-10 illustrate aspects of a method of repairing the native aortic heart valve.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

The terms “transaortic valve implantation” and “transcatheter aortic valve implantation” may be used interchangeably and may each be referred to using the acronym “TAVI”. The terms “transaortic valve replacement” and “transcatheter aortic valve replacement” may be used interchangeably and may each be referred to using the acronym “TAVR”.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. Such therapies may be highly invasive to the patient. Disclosed herein are medical devices that may be used within a portion of the cardiovascular system in order to diagnose, treat, and/or repair the system, for example during and/or in conjunction with a TAVI or TAVR procedure, or in place of a TAVI or TAVR procedure in patients not suitable for such. At least some of the medical devices disclosed herein may be delivered percutaneously and, thus, may be much less invasive to the patient, although other surgical methods and approaches may also be used. The devices disclosed herein may also provide a number of additional desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient's anatomy, such as but not limited to, arteries, veins, and/or other body lumens.

The figures illustrate selected components and/or arrangements of anatomy, a balloon valvuloplasty catheter, and/or methods of using the balloon valvuloplasty catheter. It should be noted that in any given figure, some features of the anatomy and/or the balloon valvuloplasty catheter may not be shown, or may be shown schematically, for simplicity. Additional details regarding some of the components of the anatomy and/or the balloon valvuloplasty catheter may be illustrated in other figures in greater detail. Additionally, not all instances of some elements or features may be shown in each figure for clarity.

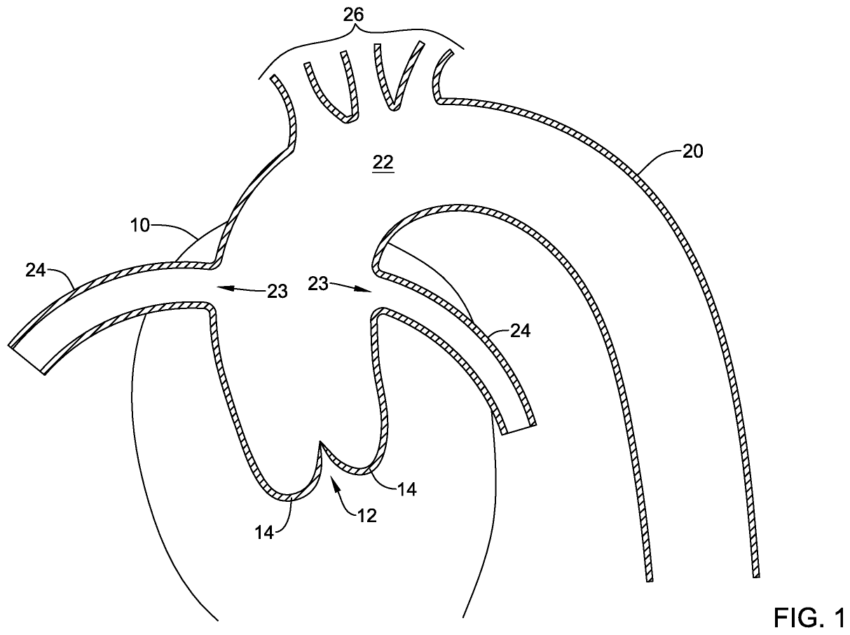

FIG. 1 illustrates a schematic partial cut-away view of a portion of a first heart 10 including the aortic valve 12 having valve leaflets 14, and certain connected vasculature, such as the aorta 20 connected to the aortic valve 12 of the first heart 10 by the aortic arch 22, the coronary arteries 24, the ostia 23 of the coronary arteries 24, and other large arteries 26 (e.g., subclavian arteries, carotid arteries, brachiocephalic artery) that extend from the aortic arch 22 to important internal organs. As mentioned above, for the purpose of this disclosure, the discussion below is directed toward use in the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

When providing treatment to a native heart valve, the native heart valve and/or the leaflets thereof may sometimes be calcified and/or subject to stenosis, which may cause and/or aggravate certain conditions. It may be beneficial, for example when a replacement heart valve implant is prescribed, to remodel the native heart valve anatomy prior to performing the procedure (e.g., TAVI, TAVR, etc.) in order to prepare the native heart valve anatomy to receive the replacement heart valve implant. One such way to remodel a native aortic valve is via a balloon aortic valvuloplasty (BAV) procedure. However, expansion of a balloon within the native aortic valve restricts blood flow through the native aortic valve. Often, such a procedure is accompanied by “rapid pacing” of the heart in order to prevent the pressure differential within the heart and/or on opposite sides of the native aortic valve from causing damage to the heart and/or other anatomy.

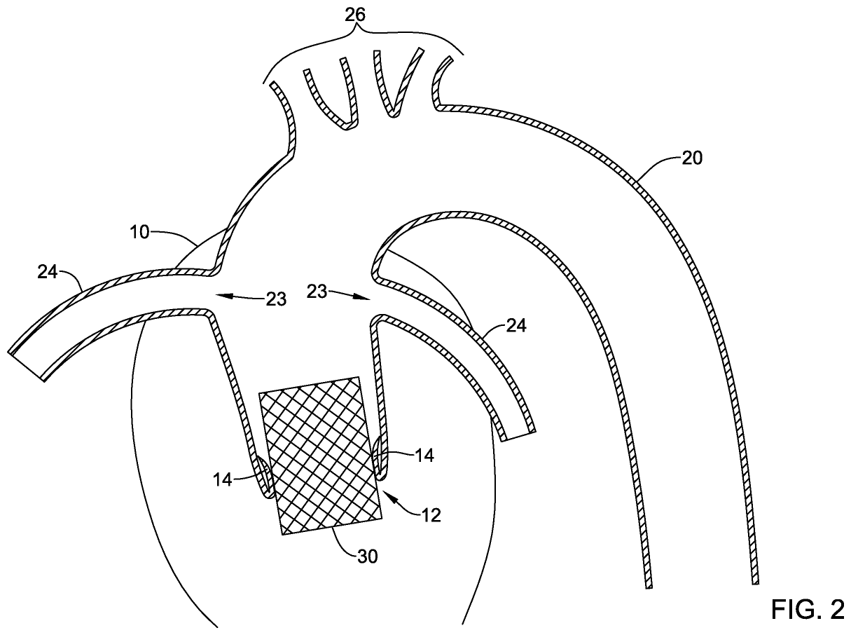

FIG. 2 illustrates the first heart 10 of FIG. 1 with a schematic example of a replacement heart valve implant 30 disposed within the aortic valve 12 of the first heart 10. In the first heart 10 illustrated in FIGS. 1 and 2, the coronary arteries 24 are spaced apart downstream from the aortic valve 12 far enough that the valve leaflets 14, which are pinched between the replacement aortic heart valve implant 30 and the wall of the aorta 20 do not impinge upon the ostia 23 of the coronary arteries 24. The first heart 10, having the configuration shown, often results in successful implantation of the replacement aortic heart valve implant 30. However, not all hearts are the same and anatomical differences may exist.

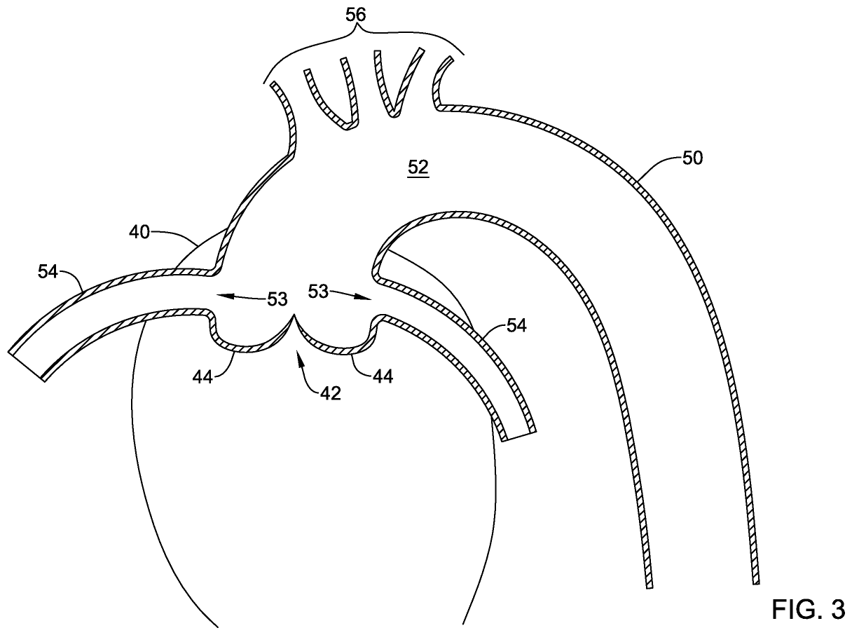

FIG. 3 illustrates a schematic partial cut-away view of a portion of a second heart 40 including the aortic valve 42 having valve leaflets 44, and certain connected vasculature, such as the aorta 50 connected to the aortic valve 42 of the second heart 40 by the aortic arch 52, the coronary arteries 54, the ostia 53 of the coronary arteries 54, and other large arteries 56 (e.g., subclavian arteries, carotid arteries, brachiocephalic artery) that extend from the aortic arch 52 to important internal organs. In the second heart 40 illustrated in FIG. 3, the coronary arteries 54 are spaced apart downstream from the aortic valve 42 a much shorter distance than the first heart 10. The second heart 40 may generally function similar to and/or the same as the first heart 10, despite the anatomical differences that may be seen in the figures. However, in some medical procedures, those anatomical differences may have a significant impact upon success or failure of the procedure.

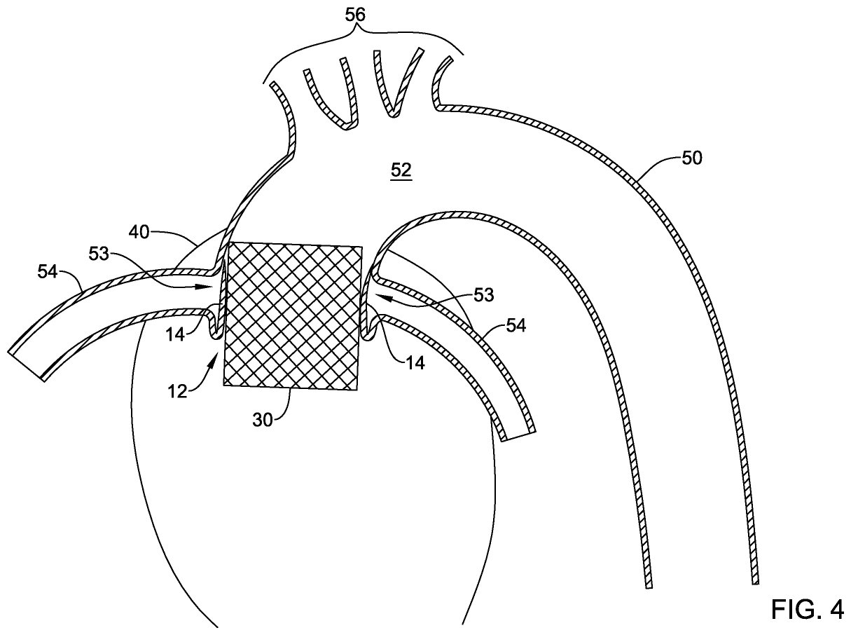

FIG. 4 illustrates the same replacement aortic heart valve implant 30 disposed within the aortic valve 42 of the second heart 40. As a result of the coronary arteries 54 being spaced apart downstream from the aortic valve 42 a much shorter distance than the first heart 10, when the valve leaflets 44 are pinched between the replacement aortic heart valve implant 30 and the wall of the aorta 50, the valve leaflets 44 impinge upon the ostia 53 of the coronary arteries 54. In anatomical configurations such as that shown in the second heart 40, implantation of the replacement aortic heart valve implant 30 and the subsequent/resulting blockage of the coronary arteries 54, even if only a partial blockage, may cause catastrophic results for the patient.

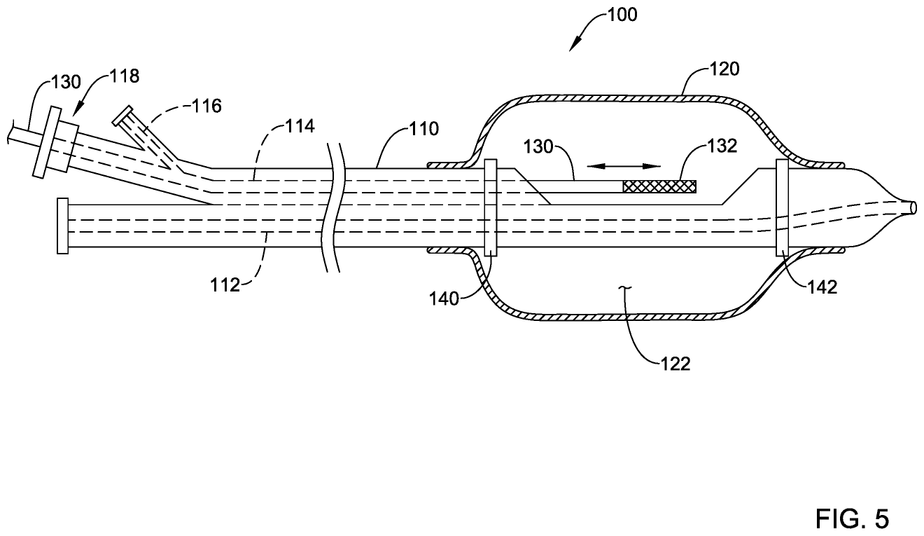

FIG. 5 illustrates a balloon valvuloplasty catheter 100 that may be used according to the methods disclosed herein, as well as others. In some embodiments, the balloon valvuloplasty catheter 100 may be used in methods of preparing a native heart valve of a patient's heart for a valve replacement procedure. In some embodiments, the balloon valvuloplasty catheter 100 may be used in method of repairing a native heart valve of a patient's heart. Other uses and procedure types are also contemplated.

The balloon valvuloplasty catheter 100 may include an elongate shaft 110 having a guidewire lumen 112 and a device lumen 114 extending longitudinally therein. In at least some embodiments, the guidewire lumen 112 may extend from a proximal end of the elongate shaft 110 to a distal end of the elongate shaft 110. In some embodiments, the guidewire lumen 112 may be sized and configured to slidably receive a guidewire therein and/or extending therethrough. In some embodiments, the guidewire lumen 112 may terminate at its proximal end at a proximal guidewire port. In some embodiments, the proximal guidewire port may be disposed at the proximal end of the elongate shaft 110. Other configurations, including those associated with single operator exchange (SOE), are also contemplated. In some embodiments, the proximal guidewire port may be disposed at a location distal of the proximal end of the elongate shaft 110.

The balloon valvuloplasty catheter 100 may include an expandable balloon 120 secured to a distal portion of the elongate shaft 110. The expandable balloon 120 may be configured to shift between a collapsed configuration and an expanded configuration. In some embodiments, the expanded configuration may be referred to as and/or interchangeably with an inflated configuration. The expandable balloon 120 may be substantially impermeable to fluids (e.g., gases, liquids, air, water, saline, blood, etc.). In some embodiments, the expandable balloon 120 may be semi-permeable and/or permeable to selected and/or pre-determined fluids (e.g., permeable to liquids but not gases, or vice versa, permeable to liquids but not semi-solids such as a gel, etc.). In some embodiments, the expandable balloon 120 may be formed from a compliant material. In some embodiments, the expandable balloon 120 may be formed from a substantially non-compliant material. The expandable balloon 120 may be configured to be expanded and/or inflated using an inflation fluid introduced into an interior 122 of the expandable balloon 120 through the elongate shaft 110.

In some embodiments, the device lumen 114 may terminate at its proximal end at a proximal device port. In some embodiments, the proximal device port may be disposed at the proximal end of the elongate shaft 110. In some embodiments, the device lumen 114 may be in fluid communication with the interior 122 of the expandable balloon 120. In some embodiments, a distal end of the device lumen 114 opens into the interior 122 of the expandable balloon 120. In some embodiments, the distal end of the device lumen 114 terminates within the interior 122 of the expandable balloon 120.

In some embodiments, the elongate shaft 110 includes an inflation lumen 116 in fluid communication with the interior 122 of the expandable balloon 120. In some embodiments, the inflation lumen 116 may terminate at an inflation port proximate the proximal end of the elongate shaft 110. In some embodiments, the device lumen 114 defines at least a portion of the inflation lumen 116. For example, within a body portion of the elongate shaft 110, the device lumen 114 and the inflation lumen 116 may be coextensive. In some embodiments, the device lumen 114 is the inflation lumen. In some embodiments, the inflation lumen 116 is fluidly connected to the device lumen 114 distal of the proximal device port.

The balloon valvuloplasty catheter 100 may include an intravascular ultrasound catheter 130 slidably disposed within the device lumen 114. In at least some embodiments, the device lumen 114 may include a proximal seal 118 configured to engage an outer surface of the intravascular ultrasound catheter 130 to thereby seal the device lumen 114 against leakage and/or contamination. In some embodiments, the intravascular ultrasound catheter 130 may include an ultrasound transducer 132 disposed proximate a distal end of the intravascular ultrasound catheter 130. In some embodiments, intravascular ultrasound catheter 130 may include the ultrasound transducer 132 disposed at the distal end of the intravascular ultrasound catheter 130. In some embodiments, the distal portion of the elongate shaft 110 may include a cutout portion proximal of a distal end of the elongate shaft 110. The device lumen 114 may end and/or terminate at the cutout portion of the elongate shaft 110. The cutout portion of the elongate shaft 110 may be disposed within the interior 122 of the expandable balloon 120. As may be seen in FIG. 5, the intravascular ultrasound catheter 130 and the ultrasound transducer 132 disposed proximate the distal end of the intravascular ultrasound catheter 130 may extend into the cutout portion of the elongate shaft 110. The ultrasound transducer 132 may be disposed within the interior 122 of the expandable balloon 120. In at least some embodiments, the ultrasound transducer 132 may be configured to translate longitudinally and/or axially within the interior 122 of the expandable balloon 120.

Intravascular ultrasound (IVUS) is a catheter-based technique that provides high-resolution, cross-sectional images of a vessel or tissue in vivo. IVUS uses ultrasound technology to see from inside blood vessels out through the surrounding blood column, visualizing the wall of the blood vessels. IVUS is sometimes used in the coronary arteries to determine the amount of atheromatous plaque built up at any particular point in the epicardial coronary artery. IVUS may also permit visualization of atheroma and/or plaque volume within the wall of the blood vessels. In some cases, IVUS can directly quantify the percentage of stenosis and give insight into the anatomy of the plaque. IVUS imaging may be performed through cannulation by a catheter with a miniature transducer that emits high-frequency ultrasound, usually in the range of 20 to 50 megahertz (MHz). As the transducer is moved through the vessel or targeted area, ultrasonic reflections are electronically converted to cross-sectional images. In some embodiments, IVUS may be used to produce a forward-looking image of the area being treated.

In some embodiments, the intravascular ultrasound catheter 130 may be configured to image tissue surrounding the expandable balloon 120 when the expandable balloon 120 is disposed in situ. In some embodiments, the intravascular ultrasound catheter 130 may be configured to image tissue surrounding the expandable balloon 120 when the expandable balloon 120 is in the expanded configuration in situ.

In some embodiments, the balloon valvuloplasty catheter 100 and/or the elongate shaft 110 may include a first radiopaque marker 140 disposed adjacent a proximal end of the expandable balloon 120 and a second radiopaque marker 142 disposed adjacent a distal end of the expandable balloon 120. In some embodiments, the first radiopaque marker 140 and/or the second radiopaque marker 142 may be fixedly attached to the elongate shaft 110. In some embodiments, the first radiopaque marker 140 and/or the second radiopaque marker 142 may be embedded within the elongate shaft 110. In some embodiments, the first radiopaque marker 140 and/or the second radiopaque marker 142 may be disposed at and/or adjacent proximal and distal ends, respectively, of the cutout portion of the elongate shaft 110. In some embodiments, the first radiopaque marker 140 and/or the second radiopaque marker 142 may be disposed within the interior 122 of the expandable balloon 120. In some embodiments, the first radiopaque marker 140 and/or the second radiopaque marker 142 may be disposed outside of the expandable balloon 120. In at least some embodiments, the first radiopaque marker 140 and the second radiopaque marker 142 may define proximal and distal limits of axial translation of the ultrasound transducer 132.

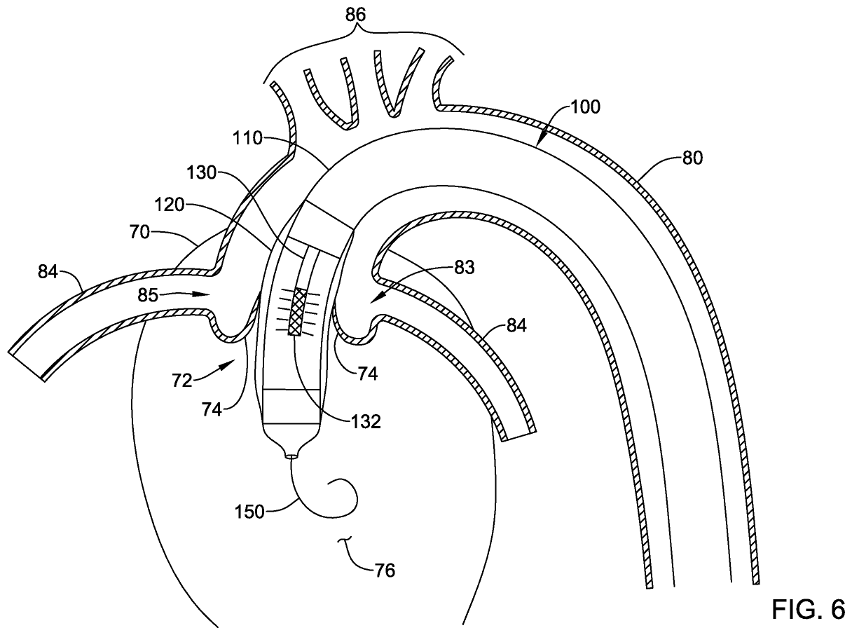

FIGS. 6-10 illustrate aspects of a method of preparing a native aortic heart valve 72 of a patient's heart 70 for transcatheter aortic valve replacement and/or a method of repairing the native aortic heart valve 72 of the patient's heart 70. As will be discussed herein, the patient's heart 70 of FIGS. 6-10 may be and/or refer to the heart 10 of FIGS. 1-2 and/or the heart 40 of FIGS. 3-4. Accordingly, the following correspondences will be appreciated: the native aortic heart valve 72 may be and/or refer to the aortic valve 12 and/or the aortic valve 42 (or in alternative embodiments, the mitral valve, the tricuspid valve, etc.); the native leaflets 74 of the native aortic heart valve 72 may be and/or refer to the valve leaflets 14 and/or the valve leaflets 44; the left ventricle 76 may be and/or refer to the left ventricle of the heart 10 and/or the heart 40; the aorta 80 may be and/or refer to the aorta 20 and/or the aorta 50; the left coronary ostium 83 and the right coronary ostium 85 of the coronary arteries 84 may be and/or refer to the left and right instances of the ostia 23 of the coronary arteries 24 and/or the ostia 53 of the coronary arteries 54; and the other large arteries 86 may be and/or refer to the other large arteries 26 and/or the other large arteries 56. The disclosed method(s) may be applied equally to either anatomical configuration, as well as others, except where expressly stated otherwise.

The method of preparing the native aortic heart valve 72 of the patient's heart 70 for transcatheter aortic valve replacement and/or the method of repairing the native aortic heart valve 72 of the patient's heart 70 may include advancing a guidewire 150 percutaneously through the native aortic heart valve 72 and into a left ventricle 76 of the patient's heart 70. The method of preparing the native aortic heart valve 72 of the patient's heart 70 for transcatheter aortic valve replacement and/or the method of repairing the native aortic heart valve 72 of the patient's heart 70 may include advancing the balloon valvuloplasty catheter 100 over the guidewire 150 to a position adjacent the native aortic heart valve 72. The method of preparing the native aortic heart valve 72 of the patient's heart 70 for transcatheter aortic valve replacement and/or the method of repairing the native aortic heart valve 72 of the patient's heart 70 may include positioning the expandable balloon 120 within the native aortic heart valve 72.

The method of preparing the native aortic heart valve 72 of the patient's heart 70 for transcatheter aortic valve replacement and/or the method of repairing the native aortic heart valve 72 of the patient's heart 70 may include imaging the left coronary ostium 83, the right coronary ostium 85, and the native leaflets 74 of the native aortic heart valve 72 using the intravascular ultrasound catheter 130. In some embodiments, imaging the left coronary ostium 83, the right coronary ostium 85, and the native leaflets 74 of the native aortic heart valve 72 using the intravascular ultrasound catheter 130 may include producing a cross-sectional or forward-looking image of the left coronary ostium 83, the right coronary ostium 85, and/or the native leaflets 74 of the native aortic heart valve 72 using the intravascular ultrasound catheter 130.

The method of preparing the native aortic heart valve 72 of the patient's heart 70 for transcatheter aortic valve replacement and/or the method of repairing the native aortic heart valve 72 of the patient's heart 70 may include inflating the expandable balloon 120 within the native aortic heart valve 72 of the patient's heart 70. In some embodiments, the method of preparing the native aortic heart valve 72 of the patient's heart 70 for transcatheter aortic valve replacement and/or the method of repairing the native aortic heart valve 72 of the patient's heart 70 may include observing via intravascular ultrasound a position of the native leaflets 74 of the native aortic heart valve 72 relative to the left coronary ostium 83 and the right coronary ostium 85.

In some embodiments, the method of preparing the native aortic heart valve 72 of the patient's heart 70 for transcatheter aortic valve replacement and/or the method of repairing the native aortic heart valve 72 of the patient's heart 70 may include adjusting a position of the intravascular ultrasound catheter 130 and/or the ultrasound transducer 132 within the interior 122 of the expandable balloon 120 by sliding the intravascular ultrasound catheter 130 axially relative to the elongate shaft 110. In some embodiments, the method of preparing the native aortic heart valve 72 of the patient's heart 70 for transcatheter aortic valve replacement and/or the method of repairing the native aortic heart valve 72 of the patient's heart 70 may include sliding the ultrasound transducer 132 axially between a proximal end of the cutout in the elongate shaft 110 and a distal end of the cutout in the elongate shaft 110. In some embodiments, the method of preparing the native aortic heart valve 72 of the patient's heart 70 for transcatheter aortic valve replacement and/or the method of repairing the native aortic heart valve 72 of the patient's heart 70 may include sliding the ultrasound transducer 132 distally within the cutout in the elongate shaft 110 and then sliding the ultrasound transducer 132 proximally within the cutout in the elongate shaft 110. In some embodiments, the method of preparing the native aortic heart valve 72 of the patient's heart 70 for transcatheter aortic valve replacement and/or the method of repairing the native aortic heart valve 72 of the patient's heart 70 may include sliding the ultrasound transducer 132 distally within the interior 122 of the expandable balloon 120 and then sliding the ultrasound transducer 132 proximally within the interior 122 of the expandable balloon 120.

In some embodiments, sliding the ultrasound transducer 132 axially within the cutout in the elongate shaft 110 and/or the interior 122 of the expandable balloon 120 may be done manually by the user. In some embodiments, sliding the ultrasound transducer 132 axially within the cutout in the elongate shaft 110 and/or the interior 122 of the expandable balloon 120 may including using a motorized translation mechanism. In some embodiments, the motorized translation mechanism may be configured to slide the ultrasound transducer 132 axially within the cutout in the elongate shaft 110 and/or the interior 122 of the expandable balloon 120 at a speed of up to 20 millimeters per second (mm/s), up to 15 mm/s, up to 12 mm/s, up to 10 mm/s, up to 7.5 mm/s, up to 5 mm/s, up to 3 mm/s, or another suitable speed commensurate with the imaging mode, intended target, and/or device capabilities.

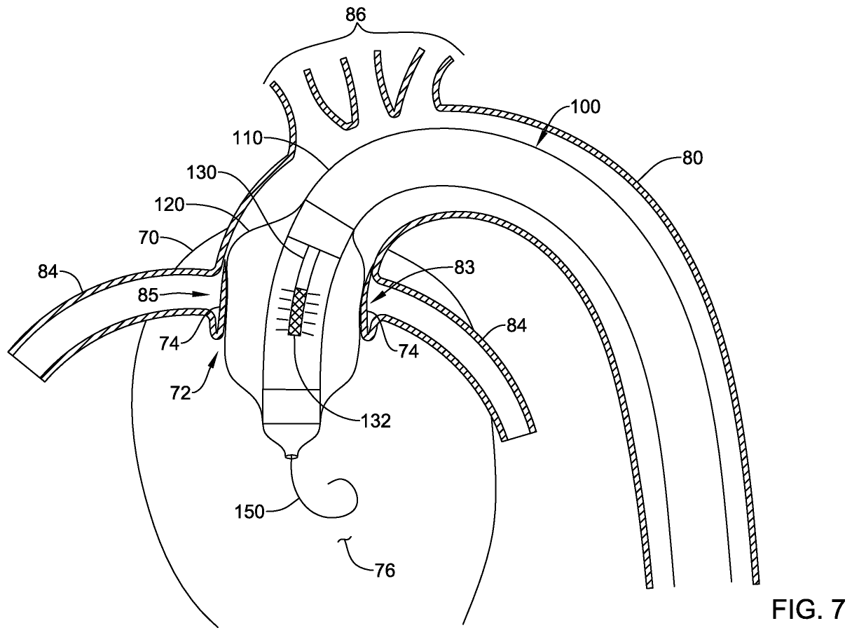

In some embodiments, the method of preparing the native aortic heart valve 72 of the patient's heart 70 for transcatheter aortic valve replacement and/or the method of repairing the native aortic heart valve 72 of the patient's heart 70 may include evaluating the position of the native leaflets 74 relative to relative to the left coronary ostium 83 and the right coronary ostium 85 to determine if the native leaflets 74 block, or at least partially block, the left coronary ostium 83 and/or the right coronary ostium 85 when the expandable balloon 120 is inflated and/or is in the expanded configuration, as seen in FIG. 7.

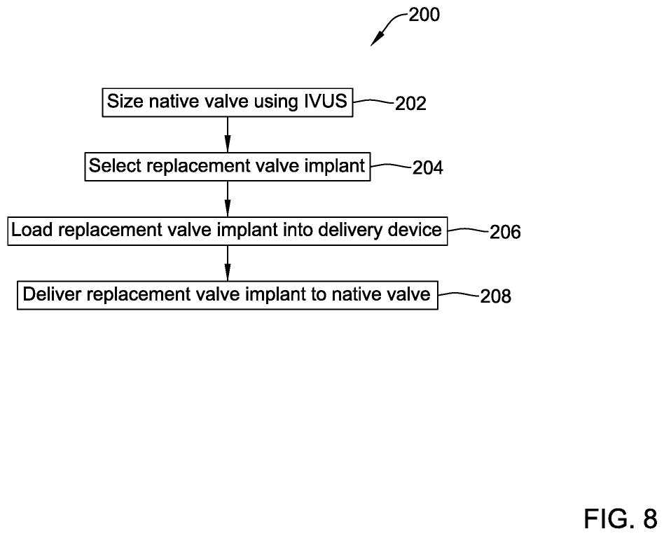

FIG. 8 illustrates aspects of a portion 200 of the method of preparing the native aortic heart valve 72 of the patient's heart 70 for transcatheter aortic valve replacement and/or the method of repairing the native aortic heart valve 72 of the patient's heart 70. In some embodiments, the method of preparing the native aortic heart valve 72 of the patient's heart 70 for transcatheter aortic valve replacement and/or the method of repairing the native aortic heart valve 72 of the patient's heart 70 may include imaging the native aortic heart valve 72 using the intravascular ultrasound catheter 130 to determine a size of the native aortic heart valve 72—see ref. 202. In some embodiments, imaging the native aortic heart valve 72 using the intravascular ultrasound catheter 130 may include three-dimensional (3D) visualization of the native aortic heart valve 72. In some embodiments, 3D visualization may be helpful to and/or may enhance diagnostic capability of the intravascular ultrasound catheter 130.

In some embodiments, imaging the native aortic heart valve 72 using the intravascular ultrasound catheter 130 occurs while inflating the expandable balloon 120 within the native aortic heart valve 72, as shown in FIG. 6. In some embodiments, imaging the native aortic heart valve 72 using the intravascular ultrasound catheter 130 occurs while the expandable balloon 120 is fully inflated and/or is in the expanded configuration within the native aortic heart valve 72, as shown in FIG. 7. In some embodiments, the method of preparing the native aortic heart valve 72 of the patient's heart 70 for transcatheter aortic valve replacement and/or the method of repairing the native aortic heart valve 72 of the patient's heart 70 may include selecting the replacement aortic heart valve implant 30 based on the size of the native aortic heart valve 72 as determined by imaging the native aortic heart valve 72 using the intravascular ultrasound catheter 130—see ref 204.

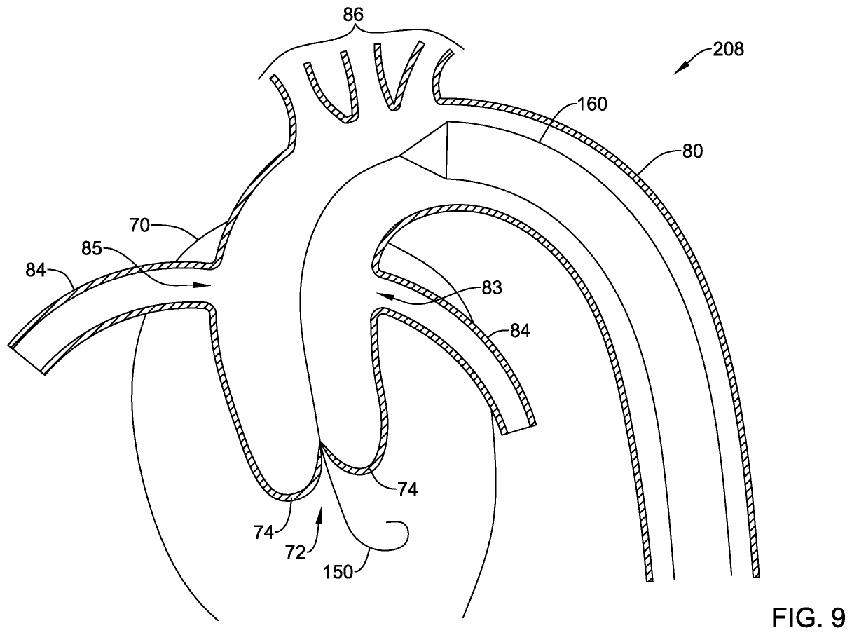

In some embodiments, the method of preparing the native aortic heart valve 72 of the patient's heart 70 for transcatheter aortic valve replacement and/or the method of repairing the native aortic heart valve 72 of the patient's heart 70 may include loading the replacement aortic heart valve implant 30 selected into a delivery device 160 (e.g., FIG. 9)—see ref 206. The method of preparing the native aortic heart valve 72 of the patient's heart 70 for transcatheter aortic valve replacement and/or the method of repairing the native aortic heart valve 72 of the patient's heart 70 may further include delivering the replacement aortic heart valve implant 30 to the native aortic heart valve 72—see ref. 208.

In some embodiments, the method of repairing the native aortic heart valve 72 of the patient's heart 70 may further include removing the balloon valvuloplasty catheter 100 while maintaining the guidewire 150 is position within the left ventricle 76 of the patient's heart 70 and in position within the aorta 80. In some embodiments, if one or both of the left coronary artery ostium 83 and the right coronary artery ostium 85 is blocked, or at least partially blocked, by the native leaflets 74 when the expandable balloon 120 is inflated and/or is in the expanded configuration, as seen in FIG. 7 for example, deployment of the replacement aortic heart valve implant 30 may be abandoned, terminated, and/or avoided.

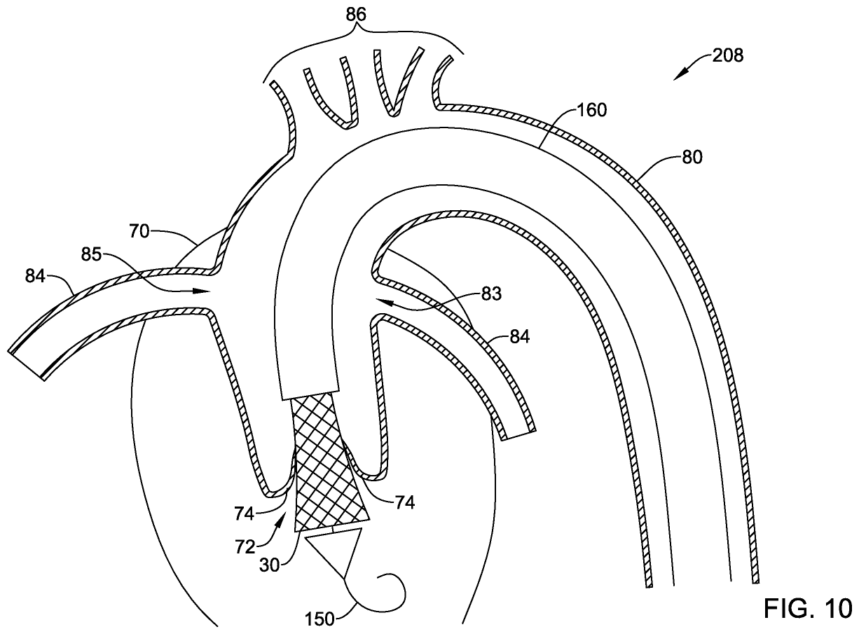

In embodiments where the method of preparing the native aortic heart valve 72 of the patient's heart 70 for transcatheter aortic valve replacement and/or the method of repairing the native aortic heart valve 72 of the patient's heart 70 may proceed (e.g., the patient's heart 70 is anatomically configured as shown in FIGS. 1-2 for example), the method(s) may include advancing the delivery device 160 percutaneously over the guidewire 150 within the aorta 80, as shown in FIG. 9, to the native aortic heart valve 72. Thereafter, the method of repairing the native aortic heart valve 72 of the patient's heart 70 may include deploying the replacement aortic heart valve implant 30 within the native aortic heart valve 72 using the delivery device 160, as seen in FIG. 10, such that neither the left coronary artery ostium 83 nor the right coronary artery ostium 85 is blocked by the native leaflets 74 when the replacement aortic heart valve implant 30 is deployed (e.g., FIG. 2).

In some embodiments, the delivery device 160 may include an outer sheath and an inner catheter disposed therein. In some embodiments, the inner catheter may extend at least partially through the outer sheath. In some embodiments, the replacement aortic valve implant 30 may be coupled to the inner catheter and disposed within the lumen of the outer sheath during delivery of the replacement aortic valve implant 30. In some embodiments, a handle may be disposed and/or attached at a proximal end of the delivery device 160 and may include one or more actuation means associated therewith. In some embodiments, the handle may be configured to manipulate the position of the outer sheath relative to the inner catheter and/or the replacement aortic valve implant 30, and/or to aid in the deployment of the replacement aortic valve implant 30. In some embodiments, the delivery device 160 may include a nose cone disposed at a distal end thereof. The delivery device 160 may be configured to slidably receive and/or slidably move over the guidewire 150. In at least some embodiments, the nose cone may have an atraumatic shape.

During delivery, the replacement aortic valve implant 30 may be generally disposed in an elongated and low profile “delivery” configuration within the outer sheath coupled to and/or distal of the inner catheter. Once positioned, the outer sheath may be retracted relative to the inner catheter and/or the replacement aortic valve implant 30 to expose the replacement aortic valve implant 30. The replacement aortic valve implant 30 may be actuated using the handle in order to translate the replacement aortic valve implant 30 into a generally expanded and larger profile “deployed” configuration (e.g., expanded but still coupled to the delivery device 160 and/or the inner catheter) suitable for implantation within the anatomy. When the replacement aortic valve implant 30 is suitably deployed within the anatomy, the replacement aortic valve implant 30 may be released and/or detached from the delivery device 160 and the delivery device 160 can be removed from the vasculature, leaving the replacement aortic valve implant 30 in place in a “released” configuration to function as, for example, a suitable replacement for the native aortic heart valve 72.

In some embodiments, the delivery device 160 may include at least one actuator element releasably connecting the replacement aortic valve implant 30 to the handle. In some embodiments, the at least one actuator element may extend distally from the inner catheter to the replacement aortic valve implant 30. In some embodiments, the at least one actuator element may be slidably disposed within and/or may extend slidably through the inner catheter. In some embodiments, the at least one actuator element may be used to actuate (i.e., translate axially or longitudinally, and/or expand) the replacement aortic valve implant 30 between the “delivery” configuration, the “deployed” configuration, and/or the “released” configuration. In some embodiments, the at least one actuator element may include a plurality of actuator elements, two actuator elements, three actuator elements, four actuator elements, or another suitable or desired number of actuator elements.

The replacement aortic valve implant 30 may include a plurality of valve leaflets (e.g., bovine pericardial, polymeric, etc.) disposed within an expandable anchor member that is reversibly actuatable between an elongated “delivery” configuration and a shortened and/or expanded “deployed” configuration. In some embodiments, the expandable anchor member may form a tubular structure defining a central longitudinal axis and a lumen extending through the expandable anchor member along, parallel to, coaxial with, and/or coincident with the central longitudinal axis from an inflow end of the expandable anchor member to an outflow end of the expandable anchor member. In some embodiments, the expandable anchor member may be and/or may include an expandable stent having a plurality of struts. In some embodiments, the expandable anchor member may be and/or include a braid formed from one or more interwoven filaments (e.g., a single filament, two filaments, etc.). In some embodiments, the expandable anchor member may be self-expanding. In some embodiments, the expandable anchor member may be expanded via mechanical means, using a balloon, or other suitable methods of expansion. Other configurations are also contemplated.

The materials that can be used for the various components of the balloon valvuloplasty catheter (and/or other elements disclosed herein) and the various components thereof disclosed herein may include those commonly associated with medical devices. For simplicity purposes, the following discussion makes reference to the balloon valvuloplasty catheter. However, this is not intended to limit the devices and methods described herein, as the discussion may be applied to other elements, members, components, or devices disclosed herein, such as, but not limited to, the elongate shaft, the expandable balloon, the intravascular ultrasound catheter, the first and second radiopaque markers, the guidewire, the delivery device, etc. and/or elements or components thereof.

In some embodiments, the balloon valvuloplasty catheter and/or other elements disclosed herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear-elastic” or “non-super-elastic” which, although may be similar in chemistry to conventional shape memory and super-elastic varieties, may exhibit distinct and useful mechanical properties. Linear-elastic and/or non-super-elastic nitinol may be distinguished from super-elastic nitinol in that the linear-elastic and/or non-super-elastic nitinol does not display a substantial “super-elastic plateau” or “flag region” in its stress/strain curve like super-elastic nitinol does. Instead, in the linear-elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear-elastic and/or non-super-elastic nitinol may also be termed “substantially” linear-elastic and/or non-super-elastic nitinol.

In some cases, linear-elastic and/or non-super-elastic nitinol may also be distinguishable from super-elastic nitinol in that linear-elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear-elastic materials such as stainless steel (that can also be distinguished based on its composition), which may accept only about 0.2 to 0.44 percent strain before plastically deforming.

In some embodiments, the linear-elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear-elastic and/or non-super-elastic nickel-titanium alloy. The mechanical bending properties of such material may therefore be generally inert to the effect of temperature over this very broad range of temperature. In some embodiments, the mechanical bending properties of the linear-elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear-elastic and/or non-super-elastic nickel-titanium alloy maintains its linear-elastic and/or non-super-elastic characteristics and/or properties.

In some embodiments, the linear-elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the balloon valvuloplasty catheter and/or other elements disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the balloon valvuloplasty catheter and/or other elements disclosed herein. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the balloon valvuloplasty catheter and/or other elements disclosed herein to achieve the same result.

In some embodiments, a degree of Magnetic Resonance Imaging (MRI) compatibility is imparted into the balloon valvuloplasty catheter and/or other elements disclosed herein. For example, the balloon valvuloplasty catheter and/or components or portions thereof may be made of a material that does not substantially distort the image and create substantial artifacts (e.g., gaps in the image). Certain ferromagnetic materials, for example, may not be suitable because they may create artifacts in an MRI image. The balloon valvuloplasty catheter or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the balloon valvuloplasty catheter and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the balloon valvuloplasty catheter and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the balloon valvuloplasty catheter and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the balloon valvuloplasty catheter and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A balloon valvuloplasty catheter, comprising:
an elongate shaft having a guidewire lumen and a device lumen extending longitudinally therein;
an expandable balloon secured to a distal portion of the elongate shaft; and
an intravascular ultrasound catheter slidably disposed within the device lumen;
wherein the device lumen is in fluid communication with an interior of the expandable balloon.

2. The balloon valvuloplasty catheter of claim 1, wherein the intravascular ultrasound catheter is configured to image tissue surrounding the expandable balloon when the expandable balloon is in an expanded configuration.

3. The balloon valvuloplasty catheter of claim 1, wherein the elongate shaft includes an inflation lumen in fluid communication with the interior of the expandable balloon.

4. The balloon valvuloplasty catheter of claim 3, wherein the device lumen defines at least a portion of the inflation lumen.

5. The balloon valvuloplasty catheter of claim 1, wherein the device lumen includes a proximal seal configured to engage an outer surface of the intravascular ultrasound catheter.

6. The balloon valvuloplasty catheter of claim 1, wherein the intravascular ultrasound catheter includes an ultrasound transducer disposed proximate a distal end of the intravascular ultrasound catheter.

7. The balloon valvuloplasty catheter of claim 6, wherein the ultrasound transducer is configured to translate longitudinally within the expandable balloon.

8. The balloon valvuloplasty catheter of claim 1, wherein the device lumen terminates within the interior of the expandable balloon.

9. The balloon valvuloplasty catheter of claim 1, wherein a first radiopaque marker is disposed adjacent a proximal end of the expandable balloon and a second radiopaque marker is disposed adjacent a distal end of the expandable balloon.

10. A method of preparing a native aortic heart valve of a patient's heart for transcatheter aortic valve replacement, comprising:
advancing a guidewire percutaneously through the native aortic heart valve and into a left ventricle of the patient's heart;
advancing a balloon valvuloplasty catheter over the guidewire to a position adjacent the native aortic heart valve;
wherein the balloon valvuloplasty catheter comprises:
an elongate shaft having a guidewire lumen and a device lumen extending longitudinally therein;
an expandable balloon secured to a distal portion of the elongate shaft; and
an intravascular ultrasound catheter slidably disposed within the device lumen, wherein the device lumen is in fluid communication with an interior of the expandable balloon;
positioning the expandable balloon within the native aortic heart valve;
imaging a left coronary artery ostium, a right coronary artery ostium, and native leaflets of the native aortic heart valve using the intravascular ultrasound catheter;
inflating the expandable balloon within the native aortic heart valve; and
observing via intravascular ultrasound a position of the native leaflets relative to the left coronary artery ostium and the right coronary artery ostium.

11. The method of claim 10, further comprising:
adjusting a position of the intravascular ultrasound catheter within the expandable balloon by sliding the intravascular ultrasound catheter axially relative to the elongate shaft.

12. The method of claim 10, further comprising:
evaluating the position of the native leaflets relative to the left coronary artery ostium and the right coronary artery ostium to determine if the native leaflets block the left coronary artery ostium and/or the right coronary artery ostium when the expandable balloon is inflated.

13. The method of claim 12, further comprising:
imaging the native aortic heart valve using the intravascular ultrasound catheter to determine a size of the native aortic heart valve.

14. The method of claim 13, wherein imaging the native aortic heart valve using the intravascular ultrasound catheter occurs while inflating the expandable balloon within the native aortic heart valve.

15. The method of claim 13, wherein imaging the native aortic heart valve using the intravascular ultrasound catheter occurs while the expandable balloon is fully inflated within the native aortic heart valve.

16. A method of repairing a native aortic heart valve of a patient's heart, comprising:
advancing a guidewire percutaneously through the native aortic heart valve and into a left ventricle of the patient's heart;
advancing a balloon valvuloplasty catheter over the guidewire to a position adjacent the native aortic heart valve;
wherein the balloon valvuloplasty catheter comprises:
an elongate shaft having a guidewire lumen and a device lumen extending longitudinally therein;
an expandable balloon secured to a distal portion of the elongate shaft; and
an intravascular ultrasound catheter slidably disposed within the device lumen, wherein the device lumen is in fluid communication with an interior of the expandable balloon;
positioning the expandable balloon within the native aortic heart valve;
imaging a left coronary artery ostium, a right coronary artery ostium, and native leaflets of the native aortic heart valve using the intravascular ultrasound catheter;
inflating the expandable balloon within the native aortic heart valve and observing via intravascular ultrasound a position of the native leaflets relative to the left coronary artery ostium and the right coronary artery ostium;
evaluating the position of the native leaflets relative to the left coronary artery ostium and the right coronary artery ostium to determine if the native leaflets block the left coronary artery ostium and/or the right coronary artery ostium when the expandable balloon is inflated;
removing the balloon valvuloplasty catheter while maintaining the guidewire in position within the left ventricle of the patient's heart;
advancing a delivery device over the guidewire to the native aortic heart valve; and
thereafter, deploying a replacement aortic heart valve implant within the native aortic heart valve using the delivery device such that neither the left coronary artery ostium nor the right coronary artery ostium is blocked by the native leaflets when the replacement aortic heart valve implant is deployed.

17. The method of claim 16, wherein if one or both of the left coronary artery ostium and the right coronary artery ostium is blocked by the native leaflets when the expandable balloon is inflated, deployment of the replacement aortic heart valve implant is abandoned.

18. The method of claim 16, further comprising:
imaging the native aortic heart valve using the intravascular ultrasound catheter while the expandable balloon is fully inflated to determine a size of the native aortic heart valve.

19. The method of claim 18, further comprising:
selecting the replacement aortic heart valve implant based on the size of the native aortic heart valve as determined by imaging the native aortic heart valve using the intravascular ultrasound catheter.

20. The method of claim 19, further comprising:
loading the replacement aortic heart valve implant selected into the delivery device.
